(19) United States
(12) Patent Application Publication
(10) Pub. No.: US 2013/0295578 A1
(43) Pub. Date: Nov. 7, 2013

(54) METHODS FOR SCREENING FOR DRUG RESISTANCE IN CANCER TREATMENT

(71) Applicants: Lorenzo F. Sempere, Lebanon, NH (US); Murray Korc, Indianapolis, IN (US)

(72) Inventors: Lorenzo F. Sempere, Lebanon, NH (US); Murray Korc, Indianapolis, IN (US)

(73) Assignee: TRUSTEES OF DARTMOUTH COLLEGE, Hanover, NH (US)

(21) Appl. No.: 13/978,724

(22) PCT Filed: Jan. 10, 2012
(86) PCT No.: PCT/US12/20740

§ 371 Date: Jul. 9, 2013

Related U.S. Application Data
(60) Provisional application No. 61/431,115, filed on Jan. 10, 2011.

(51) Int. Cl. G01N 33/50

(52) U.S. Cl. CPC G01N 33/5008
USPC 435/6.13; 435/29

(57) ABSTRACT

The present invention includes methods for screening for drug resistance in cancer tissue ex vivo which comprises a novel 3-dimensional cell culture system that mimics the tumor microenvironment.

### INTRODUCTION

This patent application claims the benefit of priority from U.S. Provisional Application Ser. No. 61/431,115, filed Jan. 10, 2011, the content of which is incorporated herein by reference in its entirety.

This invention was made with government support under Grant No. CA-R37-075059 awarded by National Institutes of Health, National Cancer Institute. The government has certain rights in the invention.

### BACKGROUND OF THE INVENTION

There is a great deal of interest in developing personalized therapy for cancer based on the knowledge that cancer is a heterogeneous group of diseases and that individual patient characteristics as well as individual tumor characteristics can affect the response of a patient to drug treatment. An example of such personalized therapy is the subtyping of breast tumor tissue for estrogen receptors before choosing what type of chemotherapy to administer, where the presence of these hormone receptors in tumor tissue are predictive of responsiveness of the cancer to endocrine therapy. Another example is the screening for epidermal growth factor receptors (EGFRs) in lung tumors in order to identify patients that can be treated with EGFR inhibitors for inhibiting tumor cell growth. For most cancers, however, such strategies for predicting tumor response to drug treatment are not available. Moreover, methods for screening tumor tissue for the propensity to develop drug resistance are also sought. Currently there are no sensitive diagnostic markers that allow for early detection or for monitoring the effectiveness of certain types of treatment and recurrence of cancer.

Pancreatic ductal adenocarcinoma (PDAC) is associated with late clinical presentation along with rapid tumor growth and early propensity for cancer cells to metastasize, factors that hinder treatment efforts. Despite advances in the understanding of the molecular mechanisms contributing to this malignancy, non-surgical therapeutic interventions have yielded little improvement in overall patient survival (Preis, M. and M. Korc. 2010. Cancer Biol. Ther. 10:9). Gemcitabine, a nucleoside analogue, may prolong life by about 8 weeks, whereas addition of the tyrosine kinase inhibitor erlotinib to gemcitabine as first-line PDAC treatment increases survival by an additional 12 days (Moore et al. 2007. J. Clin. Oncol. 25:1960-1966). New targeted approaches and assays for preclinical validation are needed.

The biological aggressiveness of PDAC is due, in part, to the presence of multiple molecular alterations, including expression of mutated KRAS (95%) and loss of tumor suppressor genes P16/CDKN2A (90%), TP53 (50-75%) and Smad4/DPC4 (40-55%) (Maitra et al. 2007. Ann. Rev. Pathol. 3:157-88). In addition, pancreatic cancer cells express high levels of the epidermal growth factor receptor (EGFR) and transforming growth factor alpha (TGF-α) and other high-affinity tyrosine kinase receptors and their respective ligands (Korc, M. 2003. Mol. Cancer 7:2-8). These cancer cells thrive in a context of marked desmoplasia characterized by activation and proliferation of fibroblasts and pancreatic stellate cells as well as foci of inflammatory cells (Korc, M. 2007. Am. J. Surg. 194:S84-S86). These stromal elements respond to cancer cell-secreted growth factors, including transforming growth factor-β (TGF-β). Indeed, cancer cells have been shown to express high levels of all three TGF-β isoforms (TGF-β1, TGF-β2, TGF-β3) and elevated TGF-β immunoreactivity in resected PDACs has been correlated with shorter overall patient survival (Friess et al. 1993. Gastroenterology 105:1846-1856). These in vivo growth promoting effects toward cancer cells have been attributed to the paracrine actions of TGF-β isoforms, as underscored by the use of a soluble TGF-β receptor strategy that sequesters cancer-derived TGF-β isoforms (Rowland-Goldsmith et al. 2002. Mol. Cancer Ther. 1:161-167; Rowland-Goldsmith et al. 2001. Clin. Cancer Res. 7:2931-2940). Moreover, TGF-β is a potent activator of pancreatic stellate cells, and the resultant reactive stroma stores and releases growth factors to the cancer cells, and produces growth factors (Korc, M. 2007. Am. J. Surg. 194:S84-S86; Vonlaufen et al. 2008. Cancer Res. 68:7707-7710). In addition to participation in autocrine and paracrine signaling these stromal elements produce a modified extracellular matrix (ECM) that further promotes cancer cell growth and metastasis (Vonlaufen et al. 2008. Cancer Res. 68:7707-7710; Mahadevan and Von Hoff. 2007. Mol. Cancer Ther. 6:1186-1197).

TGF-β effects are known to be cell type-specific and context-dependent. TGF-β represses normal epithelial cell growth, stimulates growth of mesenchymal and endothelial cells, suppresses cancer cell-directed immune mechanisms, and supports advanced stage cancer cell growth (Biere and Moses. 2006. Nat. Rev. Cancer 6:506-520; Ikushima and Miyazono. 2010. Nat. Rev. Cancer 10:415-424). TGF-β signaling is mediated by a network of Smad-dependent and Smad-independent signaling pathways that transduce TGF-β stimuli from the activated heterotetrameric TGF-β type I and type II receptor (TGFβRI/II) complex (Biere and Moses. 2006. Nat. Rev. Cancer 6:506-520; Ikushima and Miyazono. 2010. Nat. Rev. Cancer 10:415-424). The frequent mutation of TGF-β signal mediator Smad4 in PDAC lesions suggests a tumor suppressive role of Smad-dependent TGF-β signaling in cancer initiation (Maitra and Hruban. 2007. Ann. Rev. Pathol. 3:157-88). This role is supported by enhanced progression of K-Ras-driven mouse models of PDAC with homozygous deletion of Smad4 or TIIβR genetic locus (Bardeesy et al. 2006. Genes Dev. 20:3130-3146; Ijchi et al. 2006. Genes Dev. 20:3147-3160; Izeradjene et al. 2007. Cancer Cell 11:229-243).

Several approaches for interfering with TGF-β signaling are currently in different stages of pre-clinical and clinical testing, and have potential to yield novel therapeutic strategies in PDAC and other cancer types (Biere and Moses. 2006. Nat. Rev. Cancer 6:506-520; Arteaga, C. L. 2006. Curr. Opin. Genet. Dev. 16:30-37; Flavell et al. 2010. Nat. Rev. Immunol. 10:554-567). However, in vitro studies suggest that pancreatic cancer cell lines are either growth inhibited by or fail to respond to TGF-β. Therefore, blocking TGF-β signaling could be potentially detrimental in PDAC cases in which cancer cell growth is repressed by TGF-β.

### SUMMARY OF THE INVENTION

The present invention is an ex vivo method of screening tumor tissue for drug resistance which comprises culturing a tumor tissue sample ex vivo by embedding the tumor tissue sample within a soft layer of growth factor-reduced extracellular matrix and overlaying the tumor tissue sample layer onto a bottom layer of solidified agar to create a 3-dimensional tissue culture system, contacting the 3-dimensional tissue culture system with a drug, and measuring the level of tumor growth in the 3-dimensional tissue culture system in the presence of the drug. In a preferred embodiment the tumor tissue sample is a tumor tissue sample from pancreas, breast, colon, lung, prostate, or ovary. Preferred embodiments may also involve use of an extracellular matrix that comprises a suspension of individual extracellular matrix components comprising collagen I, collagen IV, laminin, and fibronectin.

In further embodiments that drugs tested can include a small molecular weight inhibitor, a monoclonal antibody, a siRNA-based drug, a microRNA-based drug, a vector-based drug, an immunotoxin, a nanoparticle-based drug, or a combination thereof.

### BRIEF DESCRIPTION OF THE DRAWINGS

FIG. 1 shows a quantification of the effects of different therapeutic compounds on size (X-axis) and number (y-axis) of spheroids when cells were assessed in the instant 3D culture system. Bar graphs indicate the number of stained (viable) spheroids in each treatment.

### DETAILED DESCRIPTION OF THE INVENTION

Given the growing recognition and acceptance of the important role that tumor microenvironment plays in cancer formation and progression, a novel modification of an extracellular matrix-based three dimensional (3D) culture system has been developed that recreates stromal-tumor microenvironment interactions. This culturing system represents a significant advancement for the study of cancer cells in a context that more closely mimics the in vivo tumor microenvironment. This cell culture system provides a novel screening method for ex vivo characterization of tumors in individual cancer patients and determining the response of such tumors to single agent or combination drug treatments. This high-throughput 3D culture system can be used to rapidly assess the response of cancerous tissue biopsies to different combinations of therapeutic regimens thereby allowing for the rapid and rational selection of a specific combination of drugs for personalized and highly effective cancer therapy. The 3D culture system of the present invention is amenable to testing and screening any potential therapeutic strategy, ranging from small molecular weight inhibitors, to monoclonal antibodies, to siRNA-based, microRNA-based and vector-based therapies, immunotoxin or nanoparticle-based drug delivery, and to various combinations thereof.

The method of the present invention utilizes a combination of anchorage-independent and anchorage-dependent growth conditions within the same screening assay. This is accomplished because of a novel modification of the standard extracellular matrix-based 3D culture system (Debnath et al. 2003. Methods 30:256-268; Gutierrez-Barrera et al. 2007. Biochem. Biophys. Res. Commun. 358:698-703) that mimics anchorage-independent growth (bottom layer of solidified 1% noble agar) while allowing for interactions with components of the extracellular matrix by overlaying with cancer cells embedded within a soft layer of 3% growth factor-reduced extracellular matrix. This 3D culture system is amenable and well-suited to grow cells that would include but not be limited to human and other mammalian cell lines, primary and secondary cultures for animal models of cancer, cell preparations obtained by fine needle aspirates of the pancreas or other organs, core needle biopsies from breast or other organs, brushed exfoliated cells from lung or other organs, incisional or exiscional biopsies and surgically resected tissue from solid tumors.

Unlike culture of cells on tissue culture-treated plastic plates, this 3D system can be used to test drugs against different sizes of epithelial structures (representative of spheroid tumor masses). Unlike conventional 3D culture systems with a bedding of 100% extracellular matrix gel that encourage polarization and differentiation, the soft agar modification creates a harsher microenvironment that may expose more overtly the aggressiveness and chemoresistance of cells, and allows for testing of both growth promoting and growth-inhibitory pathways.

There is interest in therapeutic intervention targeting TGF-β signaling in cancer aimed at enabling (de-repressing) anti-tumoral immune responses. Prior work has suggested that tumor promoting effects of TGF-β in PDAC were due to paracrine action on reactive stroma (increased proliferation and fibrosis), on the vasculature (increase proliferation and neoangiogenesis) and immune cells (evasion of immune response), occurring in conjunction with loss of TGF-β-mediated inhibitory effects on the cancer cells. Previous studies provided contradictory effects of TGF-β on pancreatic cancer cell lines when assays were conducted in vitro compared to in vivo (xenograft transplantation) and genetically-engineered mouse models. Thus, concerns exist with systemic blockade of TGF-β signaling since this may be detrimental in PDAC patient cases in which TGF-β may still suppress and oppose cancer cell growth.

Using the modified extracellular matrix-based 3D culture system of the present invention, direct growth promoting effects of TGF-β have now been demonstrated, in particular positive cooperation with EGF-mediated growth, in a subset of human and murine pancreatic cancer cell lines. This rapid and high-throughput 3D culture system has also been shown to serve as an ex vivo assay for assessing the response of tumor-derived cells obtained from mouse models of PDAC. These results indicate that subgroups of PDAC patients may benefit from therapeutic regimens that concomitantly target EGF and TGF-β, and provide enablement for a novel clinical assay in which PDAC-derived biopsies can be rapidly tested for sensitivity to different therapeutic regimens, thereby facilitating targeted treatment for individual tumors.

In order to develop a system for assessing the response of tumor-derived cells to drug treatment, the effects of growth factor stimulation on pancreatic cancer cells in a system that mimics the tumor microenvironment was chosen for proof of concept. The standard extracellular matrix-based 3D culture system, such as has been described by others (Debnath et al. 2003. Methods 30:256-268; Gutierrez-Barrera et al. 2007. Biochem. Biophys. Res. Commun. 358:698-703), was modified to create a novel system that mimics anchorage-independent growth (bottom layer of solidified 1% noble agar) while allowing for interactions with components of the extracellular matrix by overlaying with pancreatic cancer cells embedded within a soft layer of 3% growth factor-reduced extracellular matrix. Pancreatic cancer cells cultured in medium without fetal bovine serum (FBS), or supplemented with 1% FBS, formed smaller structures, and accordingly the response to growth factor stimulation was of a lesser magnitude as compared to PCCs in medium with 5% FBS. The high FBS condition was used in all subsequent experiments, since more overt differences could be observed when cells were subject to different treatments, discussed below, and since in vivo cancer cells are exposed to a rich milieu replenished with growth factors, cytokines and other nutrients.

ASPC-1, BxPC3, COLO-357, T3M4 are human cell lines that are widely used to study pancreatic cancer biology. These cells harbor different mutations in K-Ras, p53, Smad4 and other etiologically relevant genes (Moore et al. 2001. Virchows Arch. 439:798-802; Sipos et al. 2003. Virchows Arch. 442:444-452). Moreover, COLO-357 and BxPC3 cells are sensitive to TGF-β-mediated growth inhibition whereas ASPC-1 and T3M4 are resistant to these growth-modulating effects of TGF-β (Baldwin et al. 1996. Int. J. Cancer 67:283-288). In addition, independent murine cell lines isolated from pancreata of K-Ras-driven mouse models were established: PK-1 and PK-2 cells from pdx1::cre; K-ras^{LSL-G1D2/+} animals and RInk-2 cells from pdx1::cre; K-ras^{LSL-G1D2/+}; p16Ink4a/p19Arf^{flox/flox}. In these mouse models, activation of mutated K-Ras^{G12D} (excision of Lox-STOP-Lox cassette) and concomitant homozygous deletion of p16Ink4/p19Arf (excision of floxed alleles) is triggered by Cre-mediated recombination within Pdx1 (pancreatic and duodenal homeobox gene)-expressing cells during embryogenesis (Maitra, A. and R. H. Hruben. 2007. Ann. Rev. Pathol. 3:157-88; Hruban et al. 2006. Cancer Res. 66:95-106). An advantage of these murine pancreatic cancer cells is that they have a well-defined mutation history that is largely dependent on the genetically-engineered activation of mutated K-Ras^{G12D} and/or loss of tumor suppressor genes (Aguirre et al. 2003. Genes Dev. 17:3112-3126; Hingorani et al. 2003. Cancer Cell 4:437-450).

To determine how pancreatic cancer cells would integrate potential interactions between EGF and TGF-β1 signals, cells were treated four days after seeding in 3% Matrigel™ with TGF-β1 at a final concentration of 100 pM and/or EGF at 1 nM. As expected, growth of pancreatic cancer cells was stimulated by EGF, which also induced ductal-like structures that were most evident in T3M4 cells. The growth of ASPC-1, BxPC3 and T3M4 was not affected by TGFB-β1 treatment. By contrast, TGF-β1 triggered profound morphological changes in TGF-β-responsive COLO-357 and murine PK-1, PK-2 and RInk-2 cells, and dramatically enhanced the growth of these murine cells. Addition of TGF-β1 did not affect the proliferative actions of EGF in ASPC-1, BxPC3 and T3M4 cells. By contrast, the combination of EGF and TGF-β1 treatment exerted dramatic growth stimulatory effects on COLO-357, PK-1, PK-2 and RInk-2 cells and further enhanced morphological changes.

To determine whether the growth promoting effects of TGF-β1 were due to direct cell-cell interactions or cell interactions with extracellular matrix components in Matrigel™, the growth of COLO-357 was assayed in parallel in soft agar and in 3D cultures. TGF-β1 decreased the size and number of colonies formed by COLO-357 cells and antagonized the growth promoting effects of EGF under anchorage-independent growth conditions in soft agar culture. Strikingly, TGF-β1 exerted mild growth stimulatory effects on its own and greatly enhanced the growth stimulatory effects of EGF in 3D culture. These results indicated that components in the extracellular matrix modulate cellular response to EGF and TGF-β1.

To further investigate the effects of extracellular matrix components on integration of growth factor signals in both human and murine pancreatic cancer cells, cellular responses were compared on conventional tissue culture-treated plastic plates and in 3% Matrigel™. Since similar effects were observed in all murine cells tested, PK-2 cells were selected as a representative line for more detailed characterization in following experiments. TGF-β1 exerted dose-dependent effects on the morphology of COLO-357 and PK-2 cells. On plastic culture, COLO-357 and PK-2 cells lines exhibited limited morphological changes at 20 pM and overt changes at 200 pM. In 3D culture, COLO-357 and PK-2 cells were more responsive to TGF-β1, and morphological changes were observed at concentration as low as 1 pM. COLO-357 and PK-2 underwent morphological changes on plastic that were consistent with an epithelial-to-mesenchymal transition (Roussos et al. 2010. Cancer Res. 70:7360-7364). Thus, cells loosened their tightly packed cobble-stone like arrangement, became more motile and acquired an elongated fibroblast-like shape. Immunostaining revealed loss of membranous E-cadherin (epithelial marker) and upregulation of intermediate filament vimentin (mesenchymal marker) exclusively in COLO-357 and PK-2, but not in T3M4 after TGF-β1 treatment on plastic. Addition of EGF slightly enhanced the TGF-β-induced effect in COLO-357 and PK-2 cells, and triggered a partial loss of E-cadherin without accompanying upregulation of vimentin expression in T3M4. These results indicate that TGF-β responsive pancreatic cancer cells are competent to undergo epithelial-to-mesenchymal transition. By contrast, immunostaining of COLO-357 and PK-2 cells growing in 3D culture revealed reduced membranous localization of E-cadherin after TGF-β1 or EGF/TGF-β1 addition, especially in cells at the periphery of the epithelial structures in 3D culture. This effect was not observed in T3M4 cells, whereas the distribution of the cytoskeleton protein tubulin (control marker) was uniform across all epithelial structures. Increased vimentin expression was not observed in these cellular structures.

Experiments were then performed to determine whether the actions of EGF and TGF-β1 in TGF-β-responsive COLO-357 and PK-2 were mediated via the TGF-β type I and II receptor (TβRI/II) complex and/or EGFR. The cells were treated with EGF and TGF-β1 after 4 days of seeding in 3D culture as well as with SB431542 (TβRI inhibitor) and/or erlotinib (EGFR tyrosine kinase inhibitor). Pre-incubation or concomitant addition of SB431542 significantly decreased EGF/TGF-β1-induced growth stimulation of COLO-357 and PK-2 cells, whereas SB431542 addition 4 days following incubation with EGF/TGF-β1 halted further growth stimulation, but did not cause regression of already formed epithelial structures. Similarly, concomitant addition of erlotinib significantly decreased EGF/TGF-β1-induced growth stimulation of COLO-357 and PK-2 cells. Incubation with both erlotinib and SB431542 had a greater inhibitory effect than either inhibitor alone. These results indicate that EGF and TGF-β1 stimuli act cooperatively in these pancreatic cancer cells through EGFR and TβRI/II signaling pathways.

To determine whether cross-talk between TβRI/II and EGFR signaling converged at a specific signaling pathway, pancreatic cancer cells were incubated with EGF/TGF-β1 in the absence or presence of LY294002 (PI3K inhibitor), U0126 (MEK1/2 inhibitor), SB203580 (p38/MAPK inhibitor) or SP600125 (JNK inhibitor). SP600125 completely blocked the stimulatory growth effects of EGF/TGF-β1 on COLO-357 cells, whereas LY294002 had a moderate inhibitory effect, and U0126 and SB203580 had a mild inhibitory effect. Conversely, LY294002 almost completely blocked the stimulatory growth effects of EGF/TGF-β1 on PK-1 and PK-2 cells, whereas U0126 had a moderate effect, SB203580 had a mild effect, and SP600125 had an almost negligible inhibitory effect. Treatment with these kinase inhibitors alone did not alter cell viability at the tested concentrations. These results indicate a requirement for JNK signaling in COLO-357 cells and for PI3K and to a lesser extent MEK1/2 signaling in murine pancreatic cancer cells in order to mediate the cooperative actions of EGF/TGF-β1.

Experiments were then performed to examine the role of EGF and TGF-β1 in chemoresistance that is observed in pancreatic cancer cells. Gemcitabine, a nucleoside analog, is currently the standard of care in PDAC treatment. Cisplatin, a DNA-crosslinker agent, has been administered in combination to gemcitabine is some treatment regimens. Enhanced chemoresistance against gemcitabine and cisplatin has been linked to mesenchymal-like and epithelial-to-mesenchymal transition characteristics of several pancreatic cancer cells (Arumugam et al. 2009. Cancer Res. 69:5820-5828; Singh et al. 2009. Cancer Cell 15:489-500). Since the effects of EGF and TGF-β1 in 3D cultures correlated with the competence of COLO-357 and PK-2 cells to undergo epithelial-to-mesenchymal transition, it was important to determine whether EGF/TGF-β1 co-treatment would protect these cells against chemotherapeutic agents. COLO-357 and PK-2 were stimulated with TGF-β1 or EGF/TGF-β1 at day 4 in 3D culture and concomitantly challenged with cisplatin or gemcitabine. Treatment with cisplatin and gemcitabine killed most COLO-357 cells after 4 days of incubation without added growth factors. This cytotoxic effect persisted over the next 4 days, as determined by MTT metabolic staining. By contrast, there were larger cellular structures in TGF-β1-treated (>5 cells) and EGF/TGF-β1-treated COLO-357 cells (>20 cells) after 4 and 8 days of incubation. Similar effects were observed in PK-2 cells, even though PK-2 cells were more chemoresistant to gemcitabine and cisplatin treatments at the tested concentrations. While concomitant treatment with SB431542 and erlotinib blocked the protective effects of EGF/TGF-β1 against gemcitabine and cisplatin in both cell types, SP600125 was markedly effective only in COLO-357 cells and U0126 was markedly effective only in PK-2 cells.

With these in vitro data demonstrating the utility of the 3D culture system to assess chemosensitivity in pancreatic cancer cell lines, experiments were then performed to determine whether the 3D culture system could be used to assess the chemosensitivity of PDAC-derived tissues ex vivo. Two well-established genetically-engineered mouse models: pdx-1::cre; K-ras^{LSL-G1D2/+}; p53^{flox/flox} (PK; p53Δ/Δ) and pdx-1::cre; K-ras^{LSL-G1D2/+}; p16Ink4a/p19Arf^{flox/flox} (PK; p16/p19Δ/Δ mice were employed (Maitra, A. and R. H. Hruben. 2007. Ann. Rev. Pathol. 3:157-88; Hruban et al. 2006. Cancer Res. 66:95-106). Minced pancreatic tissue of about 1-5 mm³ in size were obtained from normal mouse pancreata (sibling non-transgenic controls) and from pancreatic tumors that harvested from PK; p53Δ/Δ animals. While normal pancreatic tissue underwent severe atrophy, the growth and viability of cancer cells and stroma from the pancreatic tumors was sustained for at least 20 days in 3D culture. To assess tissue response to different chemotherapeutic treatments, tumor tissues were digested with collagenase to obtain single cell suspensions for accurate cell counting and equal cell seeding in replica experimental wells. Treatment with cisplatin or gemcitabine reduced the number and size of cellular structures in 3D culture from PK; p53Δ/Δ and PK; p16/p19Δ/Δ animals. Cisplatin was more effective at reducing growth and cell survival of PK; p16/p19Δ/Δ cells, whereas gemcitabine had similar cytotoxic effects on both PK; p16/p19Δ/Δ and PK; p53Δ/Δ cells. In all cases, concomitant incubation with erlotinib and SB431542 enhanced the cytotoxic effects of cisplatin and gemcitabine. These data were consistent with observations in PK-2 and other murine secondary cultures, and demonstrate that concomitant blockade of EGF and TGF-β signaling attenuates growth-promoting and chemoresistance of pancreatic cancer cells.

The tumor microenvironment plays an important role in the course of carcinogenesis. Several 3D culturing systems have been implemented to delineate potential cancer cell-stromal interactions (Debnath, J. and J. S. Brugge. 2005. Nat. Rev. Cancer 5:675-688). One commercially available extracellular matrix, Matrigel™, a collagen IV/laminin-rich extracellular matrix gel, has been used to study cell adhesion, polarity and migration as well as integration of cell signaling (Debnath, J. and J. S. Brugge. 2005. Nat. Rev. Cancer 5:675-688; Ghajar, C. M. and M. J. Bissell. 2008. Histochem. Cell Biol. 130:1105-1118). Matrigel™-based studies in breast cancer have shown that extracellular matrix components stimulate expression and signaling of integrins, EGFR and family members HER2 and HER3, and multiple MAPK pathways, and modulate cell response to targeted therapy against HER2, including humanized monoclonal antibody (trastuzumab) (Wang et al. 2010. Oncogene 29:3335-3348; Wang et al. 2009. Cancer Res. 69:475-482; Weigeit et al. 2010. Breast Cancer Res. Treat. 122:35-43). It has now been established that a 3D culture system in which anchorage-independent growth in soft agar is modulated by an overlay of 3% artificial extracellular matrix functions to mimic the extracellular matrix that exists in vivo. In addition, a MTT metabolic readout assay with high-throughput (48-well plate format) has been used to quantitate differences in cell size and viability of epithelial structures, thereby eliminating the need of sophisticated instruments for image analysis (Ghajar, C. M. and M. J. Bissell. 2008. Histochem. Cell Biol. 130:1105-1118).

Application of the 3D culturing system of the present invention has been successful in showing the intrinsic effects of TGF-β on pancreatic cancer cells and integration of EGF and TGF-β signals by these cells within the context of a reconstituted extracellular matrix. It has also been shown that TGF-β1 exerts different effects on pancreatic cancer cells cultured on plastic plates and soft agar (growth inhibition), by comparison with culture in the novel 3D culture system (growth stimulation) of the present invention. Several mechanisms may explain the observation that TGF-β1, especially in combination with EGF, increased the growth of cells in the 3D culture system which include: 1) certain extracellular matrix components may modulate the TGF-β signal; 2) interactions between pancreatic cancer cells, extracellular matrix, and soft agar may provide additional pathways for integrating TGF-β signaling in a manner that promotes proliferation; and 3) extracellular matrix components may assist in presenting TGF-β to TβRII.

It has been shown that TGF-β confers resistance to trastuzumab in BT474 breast cancer cells in 3D culture via clustering of integrins and HER2 molecules (Wang et al. 2009. Cancer Res. 69:475-482). Blockade of integrin-mediated signaling through focal adhesion kinase and Src with PP2 small molecule inhibitor prevented activation of PI3K/AKT pathway and disrupted chemoresistant effects of TGF-β in BT474 cells. In the present study, PI3K inhibition by LY294002 in murine pancreatic cancer cells, and to a lesser extent in human cells (COLO-357), interfered with cooperative growth stimulation of EGF and TGF-β1. Thus, it is possible that the molecular pathways that ultimately integrate these signals may differ between PDAC and breast cancer cells.

Smad-dependent and Smad-independent pathways can activate a variety of signaling pathways. TGF-β can activate JNK via Smad-dependent and Smad-independent mechanisms (Bierie, B. and H. L. Moses. 2006. Nat. Rev. Cancer 6:506-520; Ikushima, H. and K. Miyazono. 2010. Nat. Rev. Cancer 10:415-424; Wang et al. 2010. Oncogene 29:3335-3348; Arany et al. 2008. Oncogene 27:2507-2512). By contrast, TGF-β activates PI3K via non-canonical, Smad-independent mechanisms (Bierie, B. and H. L. Moses. 2006. Nat. Rev. Cancer 6:506-520; Ikushima, H. and K. Miyazono. 2010. Nat. Rev. Cancer 10:415-424). It has now been found that JNK inhibition prevents EGF/TGF-β mediated growth stimulation in COLO-357 cells, indicating that the JNK pathway mediates the cross-talk between EGF and TGF-β in these human cells and that this pathway is crucial for growth stimulation of these cells in 3D culture. By contrast, the growth stimulatory effects of EGF/TGF-β1 in PK-1 and PK-2 murine pancreatic cancer cells where markedly attenuated by the PI3K inhibitor LY294002. Thus, in these murine cells, the cross-talk between EGF and TGF-β pathway was dependent on non-canonical activation of PI3K.

Interactions of Smad proteins with mutated p53 and formation of a tertiary complex with p63 in the context of Kras activation has been suggested as a switch of TGF-β signaling from tumor suppressive to tumor promoting effects (Adorno et al. 2009. Cell 137:87-98). It is well-recognized that the in vivo milieu in which cancer cells are embedded is not fully recapitulated by in vitro culturing conditions. Pancreatic cancer cells established from PDAC patient biopsies required prolonged growth on plastic plates that may have selected against TGF-β dependence or altered TGF-β signaling networks. Thus, there may be different molecular subtypes of PDAC based on the intrinsic effects of TGF-β signal on pancreatic cancer cells. At one end of the spectrum there would be a TGF-β independent subtype, in which cancer cells progress early on by disengaging tumor suppressive functions of TGF-β (loss of heterozygosity of Smad4 and/or other gene silencing mechanisms) and are constrained to evolve and forfeit later tumor promoting effects of TGF-β. At the other end of the spectrum there would be a TGF-β dependent subtype, in which cancer cells progress early on bypassing tumor suppressive functions of TGF-β.

Targeted approaches to interfere with TGF-β signaling are currently being tested in clinical trials (Flavell et al. 2010. Nat. Rev. Immunol. 10:554-567). The protocols have employed TGF-β blocking antibodies, TGF-β antisense RNA molecules, TβRII soluble proteins, and small molecule inhibitors of TβRI kinase activity such as SB431542 used in this study (Bierie, B. and H. L. Moses. 2006. Nat. Rev. Cancer 6:506-520; Arteaga, C. L. 2006. Curr. Opin. Genet. Dev. 16:30-37; Flavell et al. 2010. Nat. Rev. Immunol. 10:554-567). Although it is generally accepted that interfering with TGF-β in tumors would decrease proliferation of reactive stroma and increase tumoricidal immune responses, concerns exist about intrinsic effect of TGF-β on cancer cells depending on the stage of disease progression. COLO-357 cells engineered to express TβRII soluble proteins were resistant to TGF-β induced in vitro growth inhibition, but also formed small size tumors in a xenograft mouse model (Rowland-Goldsmith et al. 2001. Clin. Cancer Res. 7:2931-294). The results provided herein with COLO-357 cells in 3D culture reconciled the apparent contradiction and uncovered the intrinsic ability of pancreatic cancer cells to be growth stimulated by TGF-β if appropriate tumor microenvironment elements are present. Moreover, it has now been shown that pancreatic lesions from mouse models of PDAC can be cultured in the 3D system of the present invention and that concomitant inhibition of EGFR and TβRI enhanced effectiveness of cisplatin and gemcitabine treatments. Together, these results support the validity of TGF-β interfering compounds as a potential treatment for certain PDAC patients.

Thus, the 3D culture system of the present invention can be utilized to culture ex vivo tumor cell preparations obtained by ultrasound-guided fine needle aspiration of a tumor mass or by surgical resection. The assay of the present invention can thus determine TGF-β dependence of cancer cell growth and assess therapeutic response of combination drug treatments. The present invention is therefore a method of establishing ex vivo functional subtypes of cancer cells from solid tumors, and provides a rational approach for developing personalized drug therapy.

Thus, the present invention is first a method of screening tumors ex vivo for drug resistance in individual cancer patients and determining the response of the tumors to single agent or combination drug treatments. This high-throughput 3D culture system can be used to rapidly assess the response of cancerous tissue biopsies to different combinations of therapeutic regimens thereby allowing for the rapid and rational selection of a specific combination of drugs for personalized and highly effective cancer therapy. The method of the present invention utilizes a combination of anchorage-independent and anchorage-dependent growth conditions within the same screening assay. The assay mimics anchorage-independent growth (bottom layer of solidified 1% noble agar) while allowing for interactions with components of the extracellular matrix by overlaying with cancer cells embedded within a soft layer of 3% growth factor-reduced extracellular matrix. The extracellular matrix may be ones commercially available, such as Matrigel™, or other extracellular matrix gel formulations or suspension of individual extracellular matrix components such as collagen I, collagen IV, laminin and/or fibronectin. Although examples have been provided showing the effect of drugs on growth of pancreatic tumor tissue samples and pancreatic cancer cells using the novel 3D system of the present invention, other tumor tissue types are contemplated and would include but not be limited to breast tumors, lung tumors, or any other organ or tissue where solid tumors arise. In fact, it is contemplated that the present method will have broad application in screening tumors for drug resistance.

The following non-limiting examples are provided to further illustrate the present invention.

### EXAMPLE 1

### Cell Culture

Human pancreatic cancer cell lines ASPC-1, BxPC3, COLO-357 and T3M4 were maintained in RPMI 1640 medium (ThermoScientific HyClone, Logan, Utah), except for COLO-357 which was maintained in DMEM (ThermoScientific HyClone), supplemented with Fetal Bovine Serum (FBS; Omega Scientific, Tarzana, Calif.), 100 units/ml penicillin and 100 μg/ml streptomycin (1× Pen/Strep; Mediatech, Manassas, Va.) in a 37° C. incubator with 20% O₂ 5% CO₂ atmosphere. The following compounds were used to treat cells in all experiments at these final concentrations: 1 nM EGF (Upstate/Millipore, Temulecula, Calif.), 100 pM TGF-β1 and 2 μM erlotinib (Genentech, Inc., South San Francisco, Calif.), 20 μM SB431542 and 10 μM SP600125 (Tocris Biosciences, Ellisville, Mo.), 10 μM LY294002 (Calbiochem, Darmstadt, Germany), 10 μM UO126 (Alexis Biochemicals, San Diego, Calif.), SB203580 (Enzo Life Sciences, Plymouth Meeting, Pa.), 10 μM cis-diamminedichloroplatinum (CDDP; Sigma, St. Louis, Mo.) and 10 nM gemcitabine (GEM; Eli Lily and Company, Indianapolis, Ind.).

### EXAMPLE 2

### 3D Culture System

An equal volume of cell suspension and ice-chilled 6% Matrigel™ (BD biosciences, San Jose, Calif.) in complete medium (RPMI 1640 supplemented with 5% FBS and 1× penicillin/streptomycin) were mixed and dispensed at: 5-15×10³ cells in 1000 μl per well of 12-well plates or 3-10×10³ cells in 200 μl per well of 48-well plates on top of a solidified 1% noble agar layer (Difco, Sparks, Md.). Ice-chilled 3% Matrigel™ in complete medium was used to replenish medium every 4 days and/or to administer compounds.

### EXAMPLE 3

### Immunostaining

Cells cultured in 3% Matrigel™ were collected 4 days after treatment on a 40 μm sieve mesh using chilled PBS to loosen Matrigel™ matrix, fixed in 10% formalin for 1 hour and coated with melted 1% noble agar. Agar plugs were fixed for up to 4 hours in formalin and paraffin-embedded in fully-automated Shandon Pathcentre instrument as described previously (Sempere et al. 2010. Clin Cancer Res. 16:4246-4255). For freshly fixed cell assays, cells were seeded at 2-5×10³ per well on 48-well plates, treated after 24 hours and collected 48 hours after treatments by rinsing with chilled PBS and fixed in 10% formalin for 30 minutes. Four μm sections of cellular structures in paraffin-embedded or freshly fixed cells were immunostained using antibody conditions and sequential tyramide signal amplification reactions as previously described (Sempere et al. 2010. Clin Cancer Res. 16:4246-4255).

### EXAMPLE 4

### Quantification of Viability

After 4 hours of staining with 2.5 mg/ml of 3-(4,5-Dimethylthiazol-2-yl)-2,5-diphenyltetrazolium bromide (MTT; Sigma) in PBS, plates were scanned and images were stored as high resolution (1200 dpi) grayscale TIFF files. Signal intensity and density was quantified using Image-Pro Plus (Media Cybernetics, Bethesda, Md.).

### EXAMPLE 5

### Mouse Husbandry and Primary Cell Culture

The K-ras^{LSL-G1D2/+} (01XJ6-B6.129-Kras2^{tm4Tyj}) and p53^{flox/flox} (FVB.129-Trp53tm1Brn) mice were obtained from the Mouse Models of Human Cancers Consortium (National Cancer Institute, Frederick, Md.). The pdx1::cre mice were obtained from G. Gu (Gu et al. 2003. Mech. Dev. 120:35-43), and the p16Ink4a/p19Arf^{flox/flox} and Smad4^{flox/flox} from N. Bardeesy (Bardeesy et al. 2006. Genes Dev. 20:3130-3146). The strains were backcrossed at least 7 generations into and/or maintained in C57 black genetic background (C57BL/6J; The Jackson Laboratory, Bar Harbor, Me.). Mouse models of PDAC were generated by breeding appropriate founder strains.

Murine pancreatic tissue from euthanized animals was mechanically minced and digested with 2 mg/ml of Collagenase Type 4 (Worthington, Lakewood, N.J.) for 1 hour at 37° C. For ex vivo primary cell culture, 10,000 cells per well of 48-well plate were immediately plated in 3% Matrigel™ as described above, except for this modification: RPMI 1640 medium was supplemented with 20% FBS, 1× penicillin/streptomycin and 25 ng/mL amphotericin B (MP Biomedicals, LLC, Solon, Ohio). For secondary cell cultures from tumor lesions of mouse models, cancer (epithelial) cells were selected by carrying out several rounds of diluted plating in RPMI 1640 medium, with a gradual decrease concentration of FBS from 20% to 5%: PK-1 and RInk-1, as previously reported (Seeley et al. 2009. Cancer Res. 69:422-430), or after subcutaneous passage in nude mice (PK-2 and RInk-2). Experiments were conducted with epithelial cells within less than 20 passages.

### EXAMPLE 6

### 3D Culturing of Cells Obtained by EUS-FNA

Endoscopic ultrasound-guided FNA needle aspiration (EUS-FNA) procedure is a commonly used tool by gastroenterologists to diagnose PDAC. Therefore, it was determined whether 3D culturing of could be used in combination with EUS-FNA. Pancreatic cell suspensions were obtained by EUS-FNA from two different patients. Cells were grown in the instant 3-dimensional tissue culture system and shown to display spheroids after 7 days in the 3D conditions. The diagnosis of both patients was confirmed to be PDAC, though the location and size of the primary tumor were different. It is contemplated that the dynamics of spheroid formation and growth could be in itself useful to confirm PDAC diagnosis and infer aggressiveness of cancer cells. Therefore, this demonstrates the feasibility of 3D culturing pancreatic cell suspension obtained by routine EUS-FNA procedure.

### EXAMPLE 7

### Assessing Drug Treatments in 3D Culture System

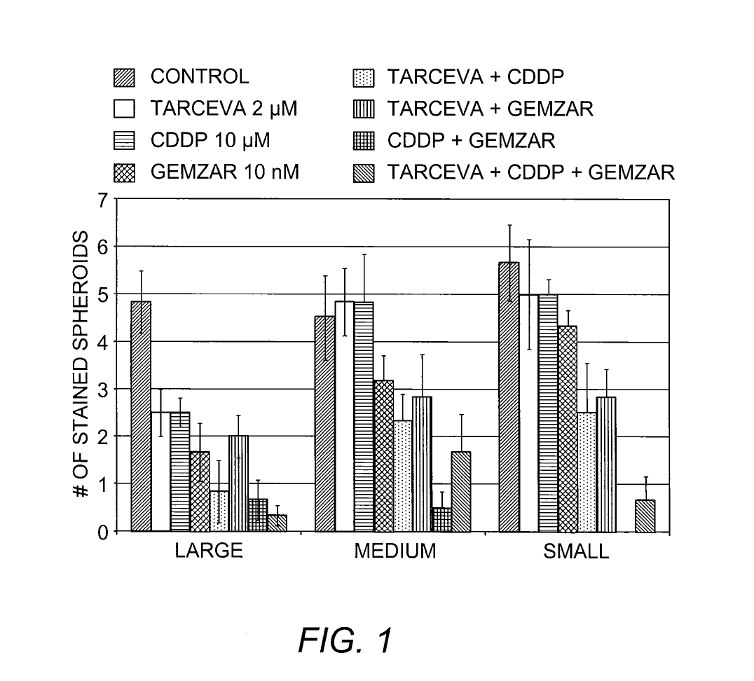

Ex vivo grown spheroids were subjected to combination drug treatments as a rapid means to assess effectiveness of the drugs. Cytostatic and/or cytotoxic effects of different therapeutic compounds currently used in the clinic for treatment of PDAC were quantified. Cell suspension from patient case FNA #144 was mixed with medium containing 6% Matrigel™ and seeded at equal volumes in multiple wells of a 48-well plate. Sets of three wells were subjected to treatment with 2 μM Tarceva™ (Erlotinib), 10 μM cis-diamminedichloro platinum (CDDP), 10 nM Gemzar™ (Gemcitabine), Tarceva™+CDDP, Tarceva™+Gemzar™, CDDP+Gemzar™, or Tarceva™+Gemzar™+CDDP. Seven days after treatment, cells were stained with MTT. The bar graphs in FIG. 1 indicate the number of stained (viable) spheroids in each treatment. Number and size of spheroids was decreased with individual compound treatments compared to untreated control cells. Further abrogation of growth was observed in samples treated with a combination of more than one compound. Therefore, the instant 3D culture system is of use as a rapid means to assess drug effectiveness.

What is claimed is:

1. An ex vivo method of screening tumor tissue for drug resistance comprising culturing a tumor tissue sample ex vivo by embedding the tumor tissue sample within a soft layer of growth factor-reduced extracellular matrix and overlaying the tumor tissue sample layer onto a bottom layer of solidified agar to create a 3-dimensional tissue culture system, contacting the 3-dimensional tissue culture system with a drug, and measuring the level of tumor growth in the 3-dimensional tissue culture system in the presence of the drug.

2. The method of claim 1, wherein said tumor tissue sample is a tumor tissue sample from pancreas, breast, colon, lung, prostate, or ovary.

3. The method of claim 1, wherein said extracellular matrix comprises a suspension of individual extracellular matrix components comprising collagen I, collagen IV, laminin, and fibronectin.

4. The method of claim 1 wherein said drug is a small molecular weight inhibitor, a monoclonal antibody, a siRNA-based drug, a microRNA-based drug, a vector-based drug, an immunotoxin, a nanoparticle-based drug, or a combination thereof.
